(19) United States
(12) Patent Application Publication
(10) Pub. No.: US 2025/0222020 A1
(43) Pub. Date: Jul. 10, 2025

(54) ORAL COMPOSITION CONTAINING DIMETHYLGLYCINE FOR IMPROVING THE CONDITION OF THE HAIR AND SCALP

(71) Applicant: DR. KURT WOLFF GMBH & CO. KG, Bielefeld (DE)

(72) Inventors: Erik SCHULZE ZUR WIESCHE, Bielefeld (DE); Maike BECKER, Bielefeld (DE); Jörn Michael VÖLKER, Bielefeld (DE)

(73) Assignee: DR. KURT WOLFF GMBH & CO. KG, Bielefeld (DE)

(21) Appl. No.: 18/854,013

(22) PCT Filed: Apr. 6, 2023
(86) PCT No.: PCT/EP2023/059231

§ 371 Date: Oct. 3, 2024

(30) Foreign Application Priority Data
Apr. 6, 2022 (DE) 10 2022 108 313.6

(51) Int. Cl. A61K 31/718; A61K 8/44; A61K 8/73; A61K 31/198; A61K 45/06; A61P 17/14; A61Q 7/00

(52) U.S. Cl. CPC A61K 31/718; A61K 8/44; A61K 8/732; A61K 31/198; A61K 45/06; A61P 17/14; A61Q 7/00

(57) ABSTRACT

The present invention relates to an oral composition which contains dimethylglycine and/or a salt of dimethylglycine and amylopectin. The present invention also relates to the (cosmetic and/or medical) use of this composition for improving the condition of the hair and/or of the scalp, for promoting the metabolism of the hair and/or of the scalp, for promoting hair growth and/or for treating and/or preventing hair loss. The present invention also relates to a nutritional supplement for promoting the metabolism of the hair and/or of the scalp and/or for promoting hair growth and/or for treating and/or preventing hair loss.

The present invention relates to an oral composition which contains dimethylglycine and/or a salt of dimethylglycine and amylopectin. The present invention also relates to the (cosmetic and/or medical) use of this composition for improving the condition of the hair and/or of the scalp, for promoting the metabolism of the hair and/or of the scalp, for promoting hair growth and/or for treating and/or preventing hair loss. The present invention also relates to a nutritional supplement for promoting the metabolism of the hair and/or of the scalp and/or for promoting hair growth and/or for treating and/or preventing hair loss.

Human hair has now largely lost its importance in protecting the body. However, healthy hair is of great cultural significance around the world for women and men, and is commonly considered a sign of wellbeing and health. As a result, thinning hair due to hair loss, for example, often has a negative impact on quality of life.

Causes of hair loss are manifold and range from age-related hair loss, through genetic or hormonal disorders, autoimmune disorders, metabolic disorders including malnutrition, to infections, trauma, tumors and drugs such as cytostatic agents and anticoagulants.

Hair loss caused by hormones such as androgens is referred to as androgenetic alopecia (alopecia androgenetica, or AGA) and, particularly in men, but also in women, is the most common cause of hair loss. A distinction can be made between male-pattern and female-pattern hair loss. Other common forms of non-scarring alopecia include telogen effluvium (TE), diffuse hair loss, as well as sudden-onset patchy hair loss caused by inflammation, which is also referred to as alopecia areata (AA).

Hair growth and hair regrowth are dependent on hair follicles receiving sufficient nutrients, which are supplied via the blood.

Decreased blood flow (microcirculation) in the scalp can therefore favor, or even cause, hair loss. In bald men, for example, the blood flow in the vertex region of the scalp is significantly lower than in men with a full head of hair. This significantly reduced microcirculation may therefore provide an explanation for hair loss as well as the failed regrowth of the hair (transition from telogen to anagen), for example in androgenetic alopecia. Overall, therefore, hair loss can be considered to be one of the first clinical signs of reduced blood flow in the peripheral vessels.

In addition, the influence of nutrient deficiencies on the structure and growth of the hair is being discussed. The effects on hair growth include acute telogen effluvium (TE), a known consequence of sudden weight loss or reduced protein supply, as well as diffuse alopecia which occurs in the event of niacin deficiency. Studies have also reported potential relationships between nutrient deficiency and chronic TE, androgenetic alopecia (AGA), female-pattern hair loss (FPHL) and alopecia areata (AA) (Guo E. L., Katta R. Diet and hair loss: effects of nutrient deficiency and supplement use. Dermatol Pract Concept. 2017; 7(1): 1).

Accordingly, micronutrients such as vitamins and minerals play an important, but not entirely clear, role in the normal development of the hair follicles and the function of immune cells. Therefore, numerous studies suggest a relationship between hair loss and micronutrient deficiency.

A review of the roles of vitamins and minerals such as vitamin A, vitamin B, vitamin C, vitamin D, vitamin E, iron, selenium and zinc in non-scarring alopecia is provided by Almohanna et al. (Almohanna, Hind M et al. “The Role of Vitamins and Minerals in Hair Loss: A Review.” Dermatology and therapy vol. 9,1 (2019): 51-70).

Guo and Katta describe the current state of studies relating to a deficiency in, or supplementation with, iron, zinc, niacin, fatty acids, selenium, vitamin D, vitamin A, vitamin E, folic acid, biotin, amino acids and proteins, and antioxidants.

Similarly, a review article by Goldberg and Lenzy discusses changes in the hair due to general malnutrition and deficiencies in micronutrients among specific patient groups (Goldberg L. J., Lenzy Y. Nutrition and hair. Clin Dermatol. 2010 July-August; 28(4):412-9).

Finally, a potential role of nutritional supplements such as amino acids, vitamins and trace elements, essential fatty acids, coenzymes Q and A, bioflavonoids, inositol, methylsulfonylmethane (MSM), dimethylglycine (DMG), silica, kelp, green tea extract, saw palmetto extract and grapeseed extract in the prevention of hair loss is described in a review article by Jain et al. (Jain, Pushpendra & Joshi, Himanshu & Das, Debajyoti. (2012). Drug that Causes Hair Loss and Promotes Hair Growth-A Review. International Journal of Research in Pharmaceutical and Biomedical Sciences. 3. 1476-82).

A deficiency in such micronutrients could be a modifiable risk factor for the development, prevention and treatment of alopecia.

While nutritional supplements containing vitamins and minerals are relatively inexpensive and easily accessible, their use requires prior knowledge of which vitamins and minerals are helpful in the treatment of hair loss. To date, however, only a few studies have been conducted which investigate the effect of micronutrient supplementation on hair growth in patients with micronutrient deficiencies and alopecia in order to establish a relationship between hair loss and micronutrient deficiencies, and there is even less literature regarding the effects of supplementation in subjects without nutrient deficiencies.

In addition, there are indications that overdosing with certain micronutrients, for example vitamin A, vitamin E or selenium, can actually promote hair loss (Almohanna et al.; Guo and Katta).

In summary, nutritional supplements may have both positive and negative effects on hair loss, and if no nutrient deficiency has been established, there is only scant indication of the type of supplementation which is suitable for re-establishing hair growth or preventing further hair loss.

Alongside this, to date there are only a few authorized pharmaceutical treatment options for hair loss, such as androgenetic alopecia, and the treatment options which are already available are either insufficiently effective or have unpleasant side effects. The situation is similarly bleak, for example, for treatment options for sudden-onset patchy hair loss caused by inflammation, alopecia areata (AA), and for hair loss with other causes.

Thus, there is still a need for improved treatment methods, in particular a need for compositions which can be used orally against hair loss; these compositions should exhibit no side effects or only negligible side effects. In particular, there is a need for compositions in the form of nutritional supplements which are easy to obtain and can be used simply. There is also a need for compositions which are pharmaceutical in nature and exhibit no side effects, or which are cosmetic in nature.

With this in mind, the object of the present invention was that of providing a highly compatible composition for treating the hair and scalp, in particular for preventing and/or treating hair loss. Furthermore, this composition should be able to be administered orally and should overcome the disadvantages of the compositions known from the prior art.

This object was achieved, surprisingly, by the composition according to claim 1 and the use thereof according to claim 8. Preferred embodiments are given in the dependent claims.

According to the invention, the object is achieved by providing an oral composition, which contains
a) dimethylglycine and/or a salt of dimethylglycine, and
b) amylopectin, wherein the weight ratio of the quantity of amylopectin in the composition to the total quantity of amylopectin and amylose in the composition is 0.9 to 1.0.

According to the invention, the component b) is the polysaccharide amylopectin, which can be present in the form of a starch-like composition consisting of amylopectin and amylose, with the proportion of amylopectin in the starch-like composition being at least 90%. The remaining proportion of the starch-like composition, of up to 10%, can be amylose.

According to the invention, the proportion of amylopectin to the total quantity of amylopectin and amylose is also at least 90% relative to the oral composition according to the invention. Accordingly, the proportion of amylose, if present, is up to 10% of the total quantity of amylopectin and amylose, also relative to the entire oral composition according to the invention. In other words, according to the definition of component b), it is essential to the invention that the composition contains a proportion of amylopectin that is as high as possible compared to the proportion of amylose.

Accordingly, according to the present invention, this gives a weight ratio of the quantity of amylopectin in the composition to the total quantity of amylopectin and amylose in the composition of 0.9 to 1.0, where, by definition, the weight ratio of 1.0 means that there is no amylose in the composition.

It has surprisingly been found that the composition according to the invention, having dimethylglycine and/or a salt of dimethylglycine and also one or more amylopectin(s), has excellent efficacy in treating hair loss, in particular hereditary and age-related hair loss. The composition activates the skin (of the scalp) and improves the supply of nutrients and oxygen to the skin (of the scalp) and the hair root. In addition, it is highly medically and cosmetically compatible; in particular, the composition is particularly medically compatible.

Dimethylglycine (N,N-dimethylglycine) is present in plants, animals and humans, but is only formed in very small quantities in humans. It is formed in a multi-stage biosynthesis of glycine, from choline as intermediate product, by transamination of betaine by means of betaine-homocysteine methylase.

N, N-dimethylglycine, also referred to as (dimethylamino)acetic acid, is represented by the following chemical formula 1:

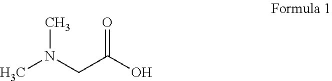

According to the present invention, it was found, surprisingly, that dimethylglycine and/or a salt of dimethylglycine is not only suitable for topical application but is also outstandingly suitable, in combination with amylopectin, as an oral preparation for improving the condition of the hair and scalp, in particular for treating or preventing hair loss.

This was particularly surprising since starches, of which the main component is amylopectin, have usually been used in the prior art as an inert carrier substance or filler, or even as a placebo in trials, and therefore no effects have been ascribed to starches or the components thereof, amylopectin and amylose (Morganti, P., et al. (1998). “EFFECT OF GELATIN-CYSTINE ANO SERENOA REPENS EXTRACT ON FREE RADICALS LEVEL ANO HAIR GROWTH.” J Appl Cosmetol 16:57-64).

It is not only the use of dimethylglycine, but also the salts, solvates and hydrates thereof, which is according to the invention. These are preferably pharmaceutically or cosmetically acceptable salts of dimethylglycine. The salt is particularly preferably a water-soluble salt having a solubility in water of at least 10 g/l at 20° C.

In a preferred embodiment, the salt of dimethylglycine is an alkali metal, alkaline-earth metal or ammonium salt of dimethylglycine.

Examples are sodium, potassium, calcium, magnesium and ammonium salts. In the ammonium salts, the ammonium cation bears one to four alkyl groups having, in each case independently of one another, 1 to 4 carbon atoms. Preference is given to the sodium and potassium salt of dimethylglycine, in particular the sodium salt of dimethylglycine, i.e. sodium dimethylglycinate (sodium N,N-dimethylglycinate).

In an alternative preferred embodiment, the salt of dimethylglycine can be the salt of an inorganic and/or organic acid with dimethylglycine.

Examples of salts of dimethylglycine with an inorganic acid are the hydrochloride, hydrobromide, hydroiodide, hydrogensulfate, sulfate, hydrogensulfite, sulfite, hydrogencarbonate, carbonate, monophosphate, diphosphate and triphosphate of dimethylglycine, and mixtures thereof. Particular preference is given to the hydrochloride of dimethylglycine, i.e. dimethylglycine hydrochloride (N,N-dimethylglycine hydrochloride). Examples of salts of dimethylglycine with an organic acid are the acetate, lactate, citrate, succinate, fumarate, maleate and benzoate of dimethylglycine, and mixtures thereof.

Preferably, according to the invention, the dimethylglycine and/or salt of dimethylglycine is selected from the group consisting of dimethylglycine, sodium dimethylglycinate and dimethylglycine hydrochloride.

It is assumed that, according to the invention, dimethylglycine and/or a salt of dimethylglycine improves cell activity and oxygen utilization in keratinocytes and therefore also promotes cell activity in the skin (of the scalp) and hair follicles. Furthermore, the skin (of the scalp) is smoothed and the skin barrier reinforced. Therefore, according to the invention, a significant hair root- and skin- (of the scalp) reinforcing effect is achieved, in particular in the treatment of ordinary or age-related stressed or weakened skin as well as hair loss, such as hereditary and age-related hair loss.

The composition according to the invention additionally contains amylopectin, wherein the weight ratio of the quantity of amylopectin in the composition to the total quantity of amylopectin and amylose in the composition is 0.9 to 1.0. Suitable weight ratios according to the invention are, in particular: 0.90; 0.91; 0.92; 0.93; 0.94; 0.95; 0.96; 0.97; 0.98; 0.99; 1.00.

In a preferred embodiment, the weight ratio of the quantity of amylopectin in the composition to the total quantity of amylopectin and amylose in the composition is 0.95 to 1.00, more preferably 0.96 to 1.00, more preferably 0.97 to 1.00, more preferably 0.98 to 1.00, more preferably 0.99 to 1.00.

Accordingly, the weight ratio of the quantity of amylopectin in the composition to the total quantity of amylopectin and amylose in the composition is 0 to 0.1. In a preferred embodiment, the weight ratio of the quantity of amylose in the composition to the total quantity of amylopectin and amylose in the composition is 0 to 0.05, more preferably 0 to 0.04, more preferably 0 to 0.03, more preferably 0 to 0.02, more preferably 0 to 0.01.

The content of amylopectin and amylose can be determined using methods known in the field. Corresponding suitable methods are for example described in the publication by Brust H, Orzechowski S, Fettke J. Starch and Glycogen Analyses: Methods and Techniques. Biomolecules. 2020; 10(7):1020.

Together with amylose, amylopectin is a component of starch.

“Starch” denotes a polysaccharide having the formula (C₆H₁₀O₅)ₙ, which consists of D-glucose units. These D-glucose units are linked to one another by glycosidic bonds, with α-1,4-glycosidic bonds forming linear chains which can be branched by means of α-1,6-glycosidic bonds. Starch usually consists of 20-30% amylose and 70-80% amylopectin.

Starch is a natural component of potatoes, cassava, cereals (e.g. wheat, maize, rice) and pulses (e.g. peas, lentils, white or red beans, chickpeas).

Amylose denotes a polysaccharide composed of α-D-glucose monomers that form linear chains having a helical structure. Amylose has a molar mass of between 100,000 and 1,000,000 g/mol, corresponding to approximately 1000 α-D-glucose monomers (in cereal starches) up to 4500 α-D-glucose monomers (in potato starches) which are α-1,4-glycosidically bonded to one another. Individual α-1,6-glycosidic branches also occur at high molar masses.

Amylopectin denotes a polysaccharide composed of α-D-glucose monomers that form highly branched structures. In this case, the α-D-glucose monomers are α-1,4-glycosidically bonded to one another. Approximately every 10-50 monomers, preferably approximately every 10-30 monomers, more preferably approximately every 10-25 monomers, more preferably approximately every 10-20 monomers, most preferably approximately every 12-15 monomers, a side chain is α-1,6-glycosidically bonded, thereby forming a tree-like branching. These side chains can be short (12 to 20 glucose units), long (30 to 45 glucose units) and very long (on average 60 glucose units) (F. A. Schüll: Einfluss spezifischer Eigenschaften der Stärke auf den Brauprozess [The influence of specific properties of starch on the brewing process], Dissertation, Technische Universität München 2012, page 12 onwards). Due to this difference in structure, amylopectin forms tangled or branched structures on a molecular level, with the branching points being predominantly on the C6 atom (Habermehl, Hammann, Krebs: Naturstoffchemie [The chemistry of natural substances] Eine Einf\ddot{u}hrung. [An introduction] 2nd edition Springer, Berlin 2002, ISBN 978-3-540-43952-3). Amylopectin has a high molar mass of 10,000,000 to 200,000,000 g/mol (C. Bächtle, P. Winkler and B. Stellbrink: Eine Frage der richtigen Stärke. [A question of the right starch] In: Chemie in unserer Zeit [Modern chemistry] 45, 250-255 (2011); F. A. Schüll: Einfluss spezifischer Eigenschaften der Stärke auf den Brauprozess [The influence of specific properties of starch on the brewing process], Dissertation, Technische Universität München 2012, page 12:3.0-4.1 million g/mol for amylopectin from barley).

The term “highly branched amylopectin” denotes a polysaccharide composed of α-D-glucose monomers having particularly highly branched structures. In this case, the α-D-glucose monomers are α-1,4-glycosidically bonded to one another, and approximately every 10-30 monomers, preferably approximately every 10-25 monomers, more preferably approximately every 10-20 monomers, most preferably approximately every 12-15 monomers, a side chain is α-1,6-glycosidically bonded, thereby forming a tree-like branching.

As used herein, the terms “amylose” and “amylopectin” denote polysaccharides consisting of α-D-glucose monomers corresponding to the above definitions, the meanings of which do not overlap.

Amylopectin can be obtained by separating amylose and amylopectin from natural plant-based starch by means of chemical, physical or enzymatic processes, for example by enzymatic treatment with amylases. The properties of amylopectin can also be modified by means of processes of this kind.

According to the invention, amylopectin is obtained from natural plant-based starch selected from potato starch (Amylum Solani), wheat starch (Amylum Tritici), rice starch (Amylum Oryzae), maize starch (Amylum Maydis), millet starch (Amylum Sorghum) or combinations thereof. In a preferred embodiment, the amylopectin used in the oral composition of the present invention is amylopectin from maize, potato or millet starch.

According to the invention, it is also possible that the amylopectin is not a waxy cereal or that the amylopectin is not obtained from a waxy cereal. In one embodiment, the amylopectin is not waxy maize or the amylopectin is not obtained from waxy maize.

According to the present invention, the amylopectin contains one or more type(s) of amylopectin which can differ in terms of the degree of branching (frequency of branches and length of side chains) and the molecular weight.

In a preferred embodiment, the amylopectin is composed of at least 1000 D-glucose monomers, preferably at least 4500, more preferably at least 5000, more preferably at least 10,000 D-glucose monomers which are α-1,4-glycosidically bonded to one another.

In a further preferred embodiment, the amylopectin contains, approximately every 10-50 monomers, preferably approximately every 10-30 monomers, more preferably approximately every 10-25 monomers, more preferably approximately every 10-20 monomers, most preferably approximately every 12-15 monomers, a side chain of D-glucose monomers which is α-1,6-glycosidically bonded.

In a further preferred embodiment, the side chains of the amylopectin comprise approximately 5-60 monomers each, preferably approximately 10-30 monomers each, more preferably approximately 10-20 monomers each, more preferably approximately 10-15 monomers each. Each of said monomers are α-D-glucose monomers.

The term “D-glucose monomers” as used herein refers to α-D-glucose monomers.

In a further preferred embodiment, the amylopectin has a molar mass of at least 100,000 g/mol, preferably of at least 100,000 g/mol to 200,000,000 g/mol, more preferably of at least 1,000,000 g/mol to 200,000,000 g/mol, more preferably of at least 10,000,000 g/mol to 200,000,000 g/mol.

The composition according to the invention may contain further polysaccharides in addition to the amylopectin b), with the proviso that the weight ratio of the quantity of amylopectin in the composition to the total quantity of amylopectin and amylose in the composition is 0.9 to 1.0.

In a preferred embodiment, the composition according to the invention does not contain any conventional starch, with the term “conventional starch” as used herein denoting starch having a composition of 20-30% amylose and 70-80% amylopectin.

In a further preferred embodiment, the composition according to the invention does not contain any amylose.

The composition according to the invention contains dimethylglycine and/or a salt of dimethylglycine preferably in a proportion of 0.1 wt % to 25.0 wt % relative to the total weight of the composition. In a preferred embodiment, the composition according to the invention contains dimethylglycine and/or a salt of dimethylglycine in a proportion of 1 wt % to 20.0 wt %, more preferably 2 wt % to 18.0 wt %, in each case relative to the total weight of the composition. In a preferred embodiment of the invention, the composition according to the invention can contain 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 20 wt % or 25 wt % dimethylglycine and/or a salt of dimethylglycine, in each case relative to the total weight of the composition.

Preferably, the composition according to the invention contains dimethylglycine and/or a salt of dimethylglycine as a pure chemical substance, including the respective solvates and hydrates thereof (e.g. the dihydrate of sodium dimethylglycinate), because this increases the purity of the composition and makes it possible to reduce the occurrence of undesired side effects. For this reason, the composition according to the invention preferably contains the chemical derivatives of dimethylglycine selected from methylglycine, trimethylglycine, (2-hydroxyethyl)trimethylammonium and trimethylhydroxybuytrobetaine at concentrations of less than 0.01 wt % relative to the total weight of the composition. The compositions according to the invention are particularly preferably entirely free of these derivatives.

In a preferred embodiment, the composition according to the invention contains c) at least one additional active ingredient, wherein the at least one additional active ingredient is selected from amla extract, amylose, amylodextrin, β-carotene, biotin, carnitine, curcumin, echinacea, ectoine, iron, folic acid, pomegranate extract, green tea extract, millet extract, millet oil, pumpkin seed oil, creatine, L-cysteine, L-lysine, niacin, niacinamide, pantolactone, pantothenic acid, piperine, procyanidine, rosemary extract, saw palmetto extract, horsetail extract, selenium, silicon, taurine, tocopheryl acetate, tomato extract, ubiquinone-10, vitamin B1, vitamin B2, vitamin B6, vitamin B12, vitamin C, vitamin E, zinc and combinations thereof, in particular selected from zinc, biotin, millet extract, selenium and combinations thereof.

Amla extract is an extract from Indian gooseberry (Phyllanthus emblica). Amla extract can be present in the composition according to the invention in a quantity of 0.1-50.0 wt %, preferably 1-30 wt %, more preferably 5-25 wt %, in each case relative to the total weight of the composition.

Amylose is preferably present in the composition according to the invention in a quantity of 0-5 wt % relative to the total weight of the composition.

Amylodextrin is a product of the conversion of starch by means of dilute acids. Amylodextrin (6 C₆H₁₀O₅+2 H₂O) is an isomeric compound of starch. Amylodextrin can also be obtained as an end product of the hydrolysis of amylopectin by β-amylase. One or more amylodextrin(s) can be present in the composition according to the invention in a quantity of 0.1-10.0 wt %, preferably 1-5 wt %, in each case relative to the total weight of the composition.

β-carotene is a precursor of vitamin A and is therefore also referred to as provitamin A. β-carotene can be present in the composition according to the invention in a quantity of 0.01-1.0 wt %, preferably 0.05-0.5 wt %, in each case relative to the total weight of the composition.

Biotin, which is also referred to as vitamin B₇ or vitamin H, is a water-soluble vitamin from the B complex. According to the invention, biotin can further reduce hair loss and strengthen the skin. Biotin can be present in the composition according to the invention in a quantity of 0.001-10.0 wt %, preferably 0.005-1.0 wt %, in each case relative to the total weight of the composition.

Carnitine can be present in the composition according to the invention in a quantity of 0.001 wt % to 10.0 wt %, preferably 0.005 wt % to 7.50 wt %, in each case relative to the total weight of the composition.

Curcumin can be present in the composition according to the invention in a quantity of 0.1-20.0 wt %, preferably 1-15 wt %, in each case relative to the total weight of the composition.

Echinacea can stimulate the circulation in the scalp and thereby supply the hair follicles with oxygen- and nutrient-rich blood, which has a further stabilizing effect on the hair and in particular on the hair root. Echinacea can be present in the composition according to the invention in a quantity of 0.001 wt % to 10.0 wt %, preferably 0.005 wt % to 7.50 wt %, in each case relative to the total weight of the composition.

Ectoine is a cyclic amino acid and, according to the invention, further stabilizes the natural structure of hair. Ectoine can be present in the composition according to the invention in a quantity of 0.001 wt % to 10.0 wt %, preferably 0.005 wt % to 7.50 wt %, in each case relative to the total weight of the composition.

Iron can be present in the composition according to the invention in a quantity of 0.01-1.0 wt %, preferably 0.05-0.5 wt %, in each case relative to the total weight of the composition.

Folic acid can be present in the composition according to the invention in a quantity of 0.001-1.0 wt %, preferably 0.05-0.5 wt %, in each case relative to the total weight of the composition.

Pomegranate extract can be present in the composition according to the invention in a quantity of 0.1-20.0 wt %, preferably 1-15 wt %, in each case relative to the total weight of the composition.

Green tea extract can be present in the composition according to the invention in a quantity of 0.1-10.0 wt %, preferably 1.0-5.0 wt %, in each case relative to the total weight of the composition.

Millet extract can be present in the composition according to the invention in a quantity of 0.01-40.0 wt %, preferably 1-35 wt %, more preferably 5-30 wt %, in each case relative to the total weight of the composition.

Millet oil can be present in the composition according to the invention in a quantity of 0.01-30.0 wt %, preferably 1-15 wt %, in each case relative to the total weight of the composition.

Pumpkin seed oil can be present in the composition according to the invention in a quantity of 0.1-20.0 wt %, preferably 1-15 wt %, in each case relative to the total weight of the composition.

Creatine can be present in the composition according to the invention in a quantity of 0.001 wt % to 10.0 wt %, preferably 0.005 wt % to 7.50 wt %, in each case relative to the total weight of the composition.

L-cysteine can be present in the composition according to the invention in a quantity of 0.01-20.0 wt %, preferably 0.1-1.0 wt %, in each case relative to the total weight of the composition.

L-lysine can be present in the composition according to the invention in a quantity of 0.1-20.0 wt %, preferably 1-15 wt %, in each case relative to the total weight of the composition.

Niacin can be present in the composition according to the invention in a quantity of 0.1-10.0 wt %, preferably 0.5-5.0 wt %, in each case relative to the total weight of the composition.

Niacinamide (also known as nicotinamide) is the amide of nicotinic acid and is also referred to as vitamin B3. In addition to other properties, for instance a reduction in oxidative stress, according to the invention, niacinamide has a hair-growth-stimulating action. Niacinamide can be present in the composition according to the invention in a quantity of 0.001 wt % to 10.0 wt %, preferably 0.005 wt % to 7.50 wt %, in each case relative to the total weight of the composition.

Pantolactone originates from the group of substituted lactones and, according to the invention, further stimulates growth factors of the hair roots. Pantolactone can be present in the composition according to the invention in a quantity of 0.001 wt % to 10.0 wt %, preferably 0.005 wt % to 7.50 wt %, in each case relative to the total weight of the composition.

Pantothenic acid can be present in the composition according to the invention in a quantity of 0.01-10.0 wt %, preferably 0.1-2.0 wt %, in each case relative to the total weight of the composition.

Piperine can be present in the composition according to the invention in a quantity of 0.01-5.0 wt %, preferably 0.05-2.0 wt %, in each case relative to the total weight of the composition.

Procyanidine can be present in the composition according to the invention in a quantity of 0.1-20.0 wt %, preferably 1-15 wt %, in each case relative to the total weight of the composition.

Rosemary extract can be present in the composition according to the invention in a quantity of 0.001-20.0 wt %, preferably 0.01-5.0 wt %, in each case relative to the total weight of the composition.

Saw palmetto extract can be present in the composition according to the invention in a quantity of 0.1-20.0 wt %, preferably 1-15 wt %, in each case relative to the total weight of the composition.

Horsetail extract can be present in the composition according to the invention in a quantity of 0.01-1.0 wt %, preferably 0.1-1.0 wt %, in each case relative to the total weight of the composition.

Selenium can be present in the composition according to the invention in a quantity of 0.0001-0.5 wt %, preferably 0.001-0.01 wt %, in each case relative to the total weight of the composition.

Silicon (silicas) can be present in the composition according to the invention in a quantity of 0.01-10.0 wt %, preferably 0.1-5.0 wt %, in each case relative to the total weight of the composition.

According to the invention, taurine, or 2-aminoethanesulfonic acid, as an antioxidant, has a further stabilizing effect on the skin and hair and in particular the hair root. Taurine can be present in the composition according to the invention in a quantity of 0.001 wt % to 10.0 wt %, preferably 0.005 wt % to 7.50 wt %, in each case relative to the total weight of the composition.

Tocopheryl acetate exhibits antioxidant properties and, according to the invention, has a further stabilizing effect on the skin and hair and in particular the hair root. Tocopheryl acetate can be present in the composition according to the invention in a quantity of 0.001 wt % to 10.0 wt %, preferably 0.005 wt % to 7.50 wt %, in each case relative to the total weight of the composition.

Tomato extract can be present in the composition according to the invention in a quantity of 0.1-20.0 wt %, preferably 1-15 wt %, in each case relative to the total weight of the composition.

Ubiquinone-10 (Q10 or coenzyme Q10) is a quinone derivative. Q10, which belongs to the ubiquinone pool, is an antioxidant and, according to the invention, has a stabilizing effect on the skin and hair and in particular the hair root. Ubiquinone-10 can be present in the composition according to the invention in a quantity of 0.001 wt % to 10.0 wt %, preferably 0.005 wt % to 7.50 wt %, in each case relative to the total weight of the composition.

Vitamin B1 (thiamine) can be present in the composition according to the invention in a quantity of 0.01-5.0 wt %, preferably 0.05-1.0 wt %, in each case relative to the total weight of the composition.

Vitamin B1 (thiamin) can be present in the composition according to the invention in a quantity of 0.01-5.0 wt %, preferably 0.05-1.0 wt %, in each case relative to the total weight of the composition.

Vitamin B2 (riboflavin) can be present in the composition according to the invention in a quantity of 0.01-5.0 wt %, preferably 0.05-1.0 wt %, in each case relative to the total weight of the composition.

Vitamin B6 can be present in the composition according to the invention in a quantity of 0.01-5.0 wt %, preferably 0.05-0.5 wt %, in each case relative to the total weight of the composition.

Vitamin B12 can be present in the composition according to the invention in a quantity of 0.00001-0.01 wt %, preferably 0.00005-0.0005 wt %, in each case relative to the total weight of the composition.

Vitamin C can be present in the composition according to the invention in a quantity of 0.1-20.0 wt %, preferably 1-15 wt %, in each case relative to the total weight of the composition.

Vitamin E can be present in the composition according to the invention in a quantity of 0.01-10.0 wt %, preferably 0.1-5.0 wt %, in each case relative to the total weight of the composition.

Zinc can be present, as a substance which has an antimicrobial action, in the composition according to the invention in a quantity of 0.01-30.0 wt %, preferably 0.1-5.0 wt %, in each case relative to the total weight of the composition.

According to the invention, the composition is administered orally. Oral administration is understood to mean internal administration.

In a preferred embodiment, the composition according to the present invention is a nutritional supplement, in particular a nutritional supplement for promoting the metabolism of the hair and/or of the scalp and/or for promoting hair growth and/or for treating and/or preventing hair loss.

Compositions according to the invention, in particular for nutritional supplements, are preferably in the form of capsules, lozenges, tablets, pills and other similar dosage forms, powder sachets, liquid ampoules, bottles with dropper inserts and similar dosage forms of liquids and powders for ingestion of the product.

In a preferred embodiment, the components, in particular a) dimethylglycine and/or a salt of dimethylglycine, b) amylopectin, wherein the weight ratio of the quantity of amylopectin in the composition to the total quantity of amylopectin and amylose in the composition is 0.9 to 1.0, and c) the other active ingredient(s) for treating the hair and/or scalp, if present, and one or more suitable carrier substance(s) in the composition according to the invention, are pressed into the form of a tablet or are present in a capsule, for example a hard gelatin capsule or a soft gelatin capsule.

Suitable carrier substances include, without being limited thereto, plant oils, such as coconut oil, soybean oil, soy lecithin, sunflower oil, olive oil, palm oil, rapeseed oil and linseed oil; beeswax; sugar alcohols such as sorbitol, mannitol, isomalt, maltitol, lactitol, xylitol and erythritol; monosaccharides such as dextrose (glucose), mannose, fructose; disaccharides such as maltose; and indigestible carbohydrates such as cellulose and pectins.

Preference is given according to the invention to using one or more plant oil(s) as carrier substances in capsules. For tablets, one or more sugars or sugar alcohols, preferably xylitol, are preferably used.

The composition according to the invention can be produced using methods known to those skilled in the art.

The present invention further relates to the use of the composition according to the invention for improving the condition of the hair and/or of the scalp and/or for promoting the metabolism of the hair and/or of the scalp and/or for promoting hair growth and/or for treating and/or preventing hair loss.

Here, the term “hair loss” covers alopecia areata (patchy hair loss), androgenetic alopecia (hereditary hair loss) in women and men, diffuse alopecia (diffuse hair loss), age-related hair loss (senescent alopecia), and hair loss caused by chemotherapy.

The hair loss to be treated is particularly preferably hereditary hair loss (androgenetic alopecia).

The hair loss to be treated is also particularly preferably age-related hair loss (senescent alopecia).

The use is in particular a cosmetic or medical use.

Surprisingly, the use according to the invention led not only to a reduction in daily hair loss, but also in addition to increased hair density, and to positive effects on hair root activity compared to a control group, with hair density and hair root activity being determined using established standard methods from the field of dermatology, before and after treatment (see inter alia Serrano-Falcön C, Fernandez-Pugnaire M A, Serrano-Ortega S. Hair and scalp evaluation: the trichogram.).

The invention is intended to be clarified using the following compositions, without wishing to limit it to these specific examples.

### EXPERIMENTAL SECTION

The following compositions were produced using measures known to those skilled in the art. The quantity of the components was chosen in each case such that the proportion by weight thereof in the premix corresponded to the stated proportions by weight.

### Example 1

Composition in the form of a tablet containing the following components:

| | | |
|---|---|---|
| Nutrient | Individual dose | Wt % |

| | | | |
|---|---|---|---|
| Vitamin C | 100 | mg | 7.28 |
| Vitamin E (α-tocopherol) | 6.8 | mg | 0.50 |
| Vitamin B2 | 1.4 | mg | 0.10 |
| Vitamin B12 | 2 | μg | 0.0001 |
| Zinc | 20 | mg | 1.46 |
| Piperine | 10 | mg | 0.73 |
| Pantothenic acid | 9 | mg | 0.66 |
| Biotin/Vitamin H | 150 | μg | 0.01 |
| Folic acid | 312 | μg | 0.02 |
| Silicon | 20 | mg | 1.46 |
| L-Cysteine | 6 | mg | 0.44 |
| Millet extract | 400 | mg | 29.12 |
| L-Lysine | 100 | mg | 7.28 |
| Na N,N-dimethylglycinate | 200 | mg | 14.56 |
| Highly branched | 500 | mg | 36.40 |

| | | |
|---|---|---|
| Amylopectin | | |
| Sugar alcohol as carrier | q.s. | q.s. |
| substance | | |

### Example 2

Composition in the form of a capsule containing the following components:

| | | |
|---|---|---|
| Nutrient | Individual dose | Wt % |

| | | | |
|---|---|---|---|
| Vitamin C | 110 | mg | 11.20 |
| Vitamin E (α-tocopherol) | 20 | mg | 2.04 |
| Niacin | 18 | mg | 1.83 |
| Zinc | 5 | mg | 0.51 |
| Pantothenic acid | 6 | mg | 0.61 |
| Natural tomato extract | 66 | mg | 6.72 |
| β-carotene/provitamin A | 2 | mg | 0.20 |
| Vitamin B6 | 1.8 | mg | 0.18 |
| Vitamin B2 (riboflavin) | 1.4 | mg | 0.14 |
| Vitamin B1 (thiamine) | 1.3 | mg | 0.13 |
| Folic acid | 312 | μg | 0.03 |
| Biotin | 150 | μg | 0.02 |
| Selenium | 32 | μg | 0.0033 |
| Vitamin B12 | 2 | μg | 0.0002 |
| N,N-Dimethylglycine | 150 | mg | 15.28 |
| hydrochloride | | | |
| Highly branched | 600 | mg | 61.10 |

| | | |
|---|---|---|
| Amylopectin | | |
| Plant oil as carrier | q.s | q.s |
| substance | | |

### Example 3

Composition in the form of a capsule containing the following components:

| Nutrient | Individual dose | Wt % |
|---|---|---|
| Biotin | 1 | 0.02 |
| Vitamin C | 100 | 1.98 |
| Vitamin E (α-tocopherol) | 7 | 0.14 |
| Zinc | 20 | 0.40 |
| Selenium | 0.2 | 0.004 |
| Iron | 8 | 0.16 |
| Silicon | 20 | 0.40 |
| Saw palmetto extract | 500 | 9.89 |
| Horsetail extract | 25 | 0.49 |
| Amla extract | 1000 | 19.79 |
| Pumpkin seed oil | 400 | 7.91 |
| Procyanidine | 400 | 7.91 |
| Green tea extract | 200 | 3.96 |
| Rosemary extract | 3 | 0.06 |
| Curcumin | 500 | 9.89 |
| Piperine | 10 | 0.20 |
| Pomegranate extract | 500 | 9.89 |
| Millet oil | 400 | 7.91 |
| L-Cysteine | 10 | 0.20 |
| L-Lysine | 200 | 3.96 |
| N,N-Dimethylglycine | 150 | 2.97 |
| hydrochloride | | |
| Highly branched | 600 | 11.87 |
| Amylopectin | | |
| Plant oil as carrier | q.s | q.s |
| substance | | |

### Reference Example 1

Composition in the form of a tablet (without Na dimethylglycinate and highly-branched amylopectin) containing the following components:

| | | |
|---|---|---|
| Nutrient | Individual dose | Wt % |

| | | | |
|---|---|---|---|
| Vitamin C | 100 | mg | 7.28 |
| Vitamin E (α-tocopherol) | 6.8 | mg | 0.50 |
| Vitamin B2 | 1.4 | mg | 0.10 |
| Vitamin B12 | 2 | μg | 0.0001 |
| Zinc | 20 | mg | 1.46 |
| Piperine | 10 | mg | 0.73 |
| Pantothenic acid | 9 | mg | 0.66 |
| Biotin/Vitamin H | 150 | μg | 0.01 |
| Folic acid | 312 | μg | 0.02 |
| Silicon | 20 | mg | 1.46 |
| L-Cysteine | 6 | mg | 0.44 |
| Millet extract | 400 | mg | 29.12 |
| Lysine | 100 | mg | 7.28 |

| | | |
|---|---|---|
| Sugar alcohol as carrier | q.s | q.s |
| substance | | |

### Study Design and Results

1. The effect of Na dimethylglycinate in combination with highly branched amylopectin was studied in the context of an in vitro investigation using human keratinocytes. To this end, HaCaT cells were cultured for 1, 3, 5 and 7 days in DMEM medium (including fetal calf serum and an antibiotic-antimycotic mix) and, inter alia, the viability and proliferation of the cells were determined using suitable measurement methods, as was the expression of the relevant growth factors for cell growth.

### Analysis of Viability:

What is referred to as an MTT assay was used for this measurement, in order to determine cell metabolic activity as an indicator of cell viability and cytotoxicity. This colorimetric assay is based on the reduction of a yellow tetrazolium salt (3-(4,5-dimethylthiazol-2-yl)-2,5-diphenyltetrazolium bromide or MTT) to purple formazan crystals by metabolically active cells.

Human epidermal keratinocytes (HaCaT) were seeded in a 96-well plate at a cell density of 5000 cells/well and cultured in medium (DMEM with 10% FBS, 1% penicillin-streptomycin, 0.5% Fungizone). On the following day (=starting point, beginning of treatment) and also after a further 24 h and 48 h and 72 h of culture at 37° C. and 5 vol % CO₂, the cell culture medium was changed without (control) or with the respectively already present active ingredient concentrations of sodium dimethylglycinate (DMG), highly-branched amylopectin and the combination of DMG and highly-branched amylopectin. The viability was measured analogously to the already-published studies in B. I. Toth, N. Dobrosi, A. Dajnoki, G. Czifra, A. Oläh, A. G. Szöllösi, I. Juhäsz, K. Sugawara, R. Paus, T. Birö, J Invest Dermatol 2011, 131, 1095-1104.

### Analysis of Proliferation:

What is referred to as a CyQUANT assay was performed for the measurement of proliferation. In this fluorescence-based assay, the fluorescent substance used binds to DNA (deoxyribonucleic acid), with the content of cellular DNA being a direct measure of the number of cells within a sample.

Human epidermal keratinocytes (HaCaT) were seeded in a 96-well plate at a cell density of 5000 cells/well and cultured in medium (DMEM with 10% FBS, 1% penicillin-streptomycin, 0.5% Fungizone). On the following day (=starting point, beginning of treatment) and also after a further 24 h and 48 h and 72 h of culture at 37° C. and 5 vol % CO₂, the cell culture medium was changed without (control) or with the respectively already present active ingredient concentrations of sodium dimethylglycinate (DMG), highly-branched amylopectin and the combination of DMG and highly-branched amylopectin. The proliferation was measured analogously to the already-published studies in A. Oläh, B. I. Töth, I. Borbirö, K. Sugawara, A. G. Szöllösi, G. Czifra, B. Päl, L. Ambrus, J. Kloepper, E. Camera, The Journal of clinical investigation 2014, 124, 3713-3724.

### Analysis of Gene Expression:

Human epidermal keratinocytes (HaCaT) were seeded in a 6-well plate at a cell density of 140,000 cells/well and cultured in medium (DMEM with 10% FBS, 1% penicillin-streptomycin, 0.5% Fungizone) at 37° C. and 5 vol % CO₂. On the following day (=starting point, beginning of treatment), the cell culture medium was changed without (control) or with the respectively already present active ingredient concentrations of sodium dimethylglycinate (DMG), highly-branched amylopectin and the combination of DMG and highly-branched amylopectin, and the cells were harvested after 24 h. Gene expression was measured using qRT-PCR on the basis of already-published studies in B. V. Diaz, M.-C. Lenoir, A. Ladoux, C. Frelin, M. Demarchez, S. Michel, Journal of Biological Chemistry 2000, 275, 642-650.

It was found that, with Na dimethylglycinate in combination with highly-branched amylopectin, the parameters relevant for growth of the HaCaT cells were positively influenced, and the expression of growth factors (for example HGF, KGF, VEGF, GM-CSF) was significantly increased compared to the treatment of the HaCaT cells with just Na dimethylglycinate or just highly-branched amylopectin.

2. The composition according to the invention from example 1 (verum-composition with sodium dimethyglycinate and highly-branched amylopectin) and the composition from reference example 1 (placebo composition without sodium dimethylglycinate and highly-branched amylopectin) were additionally administered to a group of test subjects of a total of 60 men and women for 6 months in the context of a randomized controlled blinded trial. The test subjects were questioned for a subjective assessment of the reversal of their hair loss after the end of the treatment, with these results being obtained by means of questionnaires. At the same time, the hair density and hair root activity were determined before and after treatment using established standard methods from the field of dermatology (see inter alia Serrano-Falcön C, Fernandez-Pugnaire M A, Serrano-Ortega S. Hair and scalp evaluation: the trichogram.). During and after the oral administration of the composition according to the invention from example 1, more test subjects stated that they had lost less hair compared to the control group who were receiving the placebo composition orally. In the context of the objective measurements, it was observed that, by oral administration of the composition according to the invention from example 1, an increase in hair density and positive effects on hair root activity were observed compared to the control group.

1. An oral composition, containing
a) dimethylglycine and/or a salt of dimethylglycine, and
b) amylopectin, wherein the weight ratio of the quantity of amylopectin in the composition to the total quantity of amylopectin and amylose in the composition is 0.9 to 1.0.

2. The composition according to claim 1, wherein the amylopectin is composed of D-glucose monomers which are α-1,4-glycosidically bonded to one another and wherein a side chain of D-glucose monomers is α-1,6-glycosidically bonded approximately every 10-30 monomers.

3. The composition according to claim 2, wherein the side chains of the amylopectin comprise approximately 5-60 monomers each, preferably approximately 10-30 monomers each, more preferably approximately 10-20 monomers each, more preferably approximately 10-15 monomers each.

4. The composition according to one of the preceding claims, wherein the amylopectin has a molar mass of at least 100,000 g/mol.

5. The composition according to one of the preceding claims, wherein the composition contains 0.1 to 25.0 wt % of dimethylglycine and/or a salt of dimethylglycine.

6. The composition according to one of the preceding claims, wherein the composition additionally contains
c) at least one additional active ingredient selected from amla extract, amylose, amylodextrin, β-carotene, biotin, carnitine, curcumin, echinacea, ectoine, iron, folic acid, pomegranate extract, green tea extract, millet extract, millet oil, pumpkin seed oil, creatine, L-cysteine, L-lysine, niacin, niacinamide, pantolactone, pantothenic acid, piperine, procyanidine, rosemary extract, saw palmetto extract, horsetail extract, selenium, silicon, taurine, tocopheryl acetate, tomato extract, ubiquinone-10, vitamin B1, vitamin B2, vitamin B6, vitamin B12, vitamin C, vitamin E, zinc and combinations thereof.

7. The composition according to one of the preceding claims, wherein the composition is a nutritional supplement for promoting the metabolism of the hair and/or of the scalp and/or for promoting hair growth and/or for treating and/or preventing hair loss.

8. A use of the composition according to one of claims 1 to 7 for improving the condition of the hair and/or of the scalp and/or for promoting the metabolism of the hair and/or of the scalp and/or for promoting hair growth and/or for treating and/or preventing hair loss.

9. The use according to claim 8, wherein the hair loss is selected from alopecia areata (patchy hair loss), androgenetic alopecia (hereditary hair loss) in women and men, diffuse alopecia (diffuse hair loss), age-related hair loss (senescent alopecia), and hair loss caused by chemotherapy.

10. The use according to claim 8 or 9, wherein the use is a medical or cosmetic use.
